(19) United States
(12) Patent Application Publication
(10) Pub. No.: US 2024/0076667 A1
(43) Pub. Date: Mar. 7, 2024

(54) APPLICATION OF ANTISENSE OLIGONUCLEOTIDE TARGETING PBX1 PROMOTER REGION G-QUADRUPLEX IN PREPARATION OF MEDICINE FOR TREATING MELANOMA

(71) Applicant: JILIN UNIVERSITY, Changchun City (CN)

(72) Inventors: Jinyu LIU, Changchun City (CN); Yutong SUI, Changchun City (CN)

(21) Appl. No.: 18/349,353

(22) Filed: Jul. 10, 2023

(30) Foreign Application Priority Data
Aug. 25, 2022 (CN) 202211023450.3

(51) Int. Cl. C12N 15/113; G01N 33/50

(52) U.S. Cl. CPC C12N 15/113; G01N 33/5011; C12N 2310/11

(57) ABSTRACT

The present invention disclosed an application of an antisense oligonucleotide targeting a PBX1 promoter region G-quadruplex in a preparation of a medicine for treating melanoma. The medicine for treating melanoma has one or more of following uses: inhibiting the proliferation of melanoma cell; inhibiting the migration of melanoma cell; inhibiting the invasion of melanoma cell; inhibiting the growth of melanoma; and inhibiting the lung metastasis of melanoma. The identification of the antisense oligonucleotide targeting the PBX1 promoter region G-quadruplex provided by the present invention provides a new drug target and new treatment methods and ideas for the development of a new generation of inhibiting melanoma medicines.

### RELATED APPLICATION

The present application claims priority from Chinese Application Number 202211023450.3 filed Aug. 25, 2022, the disclosure of which is hereby incorporated by reference herein in its entirety.

### INCORPORATE BY REFERENCE

The sequence listing provided in the file entitled C6896-001_Rev2.xml, which is an Extensible Markup Language (XML) file that was created on Jul. 6, 2023, and which comprises 3,522 bytes, is hereby incorporated by reference in its entirety.

### TECHNICAL FIELD

The present invention relates to the field of biomedicine, and particularly to the application of the antisense oligonucleotide targeting the PBX1 promoter region G-quadruplex in the preparation of the medicine for treating melanoma.

### BACKGROUND

Melanoma is the malignant tumor caused by melanocytes, is the most aggressive type of skin cancer, and its incidence has been increasing in recent years. According to the statistics of the American Cancer Society, melanoma is still the most deadliest type of skin tumors, with an estimated 106,110 new cases and 7,180 deaths in 2021. Although many treatment methods are developed and applied, melanoma prognosis remains poor, and therefore, there is an urgent need for further studies to identify a more effective treatment solution, for achieving better clinical effects.

### SUMMARY

In order to solve the above-mentioned deficiencies of the prior art, the present invention provides the application of the antisense oligonucleotide targeting the PBX1 promoter region G-quadruplex in the preparation of the medicine for treating melanoma. In-vivo studies show that the treatment using the antisense oligonucleotide targeting the PBX1 promoter region G-quadruplex almost completely inhibits growth and lung metastasis of melanoma. The present invention provides the new drug target and new treatment method and ideas for development of the new generation of medicine for treating melanoma.

The purpose of the present invention is implemented by the following technical solutions:

In a first aspect of the present invention, provided is the application of the antisense oligonucleotide targeting the PBX1 promoter region G-quadruplex in the preparation of the medicine for treating melanoma.

Specifically, the medicine for treating melanoma has one or more uses as follows:
inhibiting the proliferation of melanoma cell;
inhibiting the migration of melanoma cell;
inhibiting the invasion of melanoma cell;
inhibiting the growth of melanoma; and
inhibiting the lung metastasis of melanoma.

Optionally, the antisense oligonucleotide targeting the PBX1 promoter region G-quadruplex comprises at least one of the followings:
the compound specifically inhibiting PBX1-G4;
the interfering molecule specifically interfering with the expression of PBX1-G4; and
the gene editing reagent specifically knocking out PBX1.

Optionally, the compound specifically inhibiting PBX1-G4 is the antisense oligonucleotide.

Preferably, the content of the antisense oligonucleotide in the medicine is 15 mg/ml to 35 mg/ml.

Optionally, the interfering molecule specifically interfering with the expression of PBX1-G4 is the antisense oligonucleotide.

Preferably, the antisense oligonucleotide has the nucleotide sequence as shown in SEQ ID Nos: 1-2.

In a second aspect of the present invention, provided is the method of screening the medicine, where the medicine is used for preventing or treating melanoma, and the method comprises:
applying the candidate medicine to the melanoma model;
quantitatively detecting PBX1 protein in the melanoma model before and after administration; and
indicating the candidate medicine is the target medicine, if the expression level of PBX1 protein in the melanoma model is reduced after the administration, compared with before the administration.

In a third aspect of the present invention, provided is the application of the reagent in the preparation of the kit, where the reagent is used for quantitatively detecting the expression level of PBX1 protein, and the kit is used for determining the effectiveness of medicine in preventing or treating melanoma.

The present invention has the following beneficial effects:

The present invention detects the effect of PBX1-G4 on growth and lung metastasis of melanoma and discovers that the expression of PBX1 is positively correlated with the development of melanoma. The sequence of PBX1 is conserved in evolution and is same in mice and human beings. Results of human (A375 cell line) and mouse (B16-F10 cell line) in vitro models show that the adjustment on the melanoma cell line by PBX1 can be translated to human pathophysiology. Studies in the present application show that the antisense oligonucleotide targeting the PBX1 promoter region G-quadruplex obviously inhibits proliferation, migration and invasion capabilities of the melanoma cell line. In vivo studies show that treatment using the antisense oligonucleotide targeting the PBX1 promoter region G-quadruplex almost completely inhibits the growth and the lung metastasis of melanoma. The identification of the present invention on the antisense oligonucleotide targeting the PBX1 promoter region G-quadruplex provides the new drug target and new treatment methods and ideas for development of the new generation of medicine for treating melanoma.

### BRIEF DESCRIPTION OF THE DRAWINGS

To describe the technical solutions of embodiments of the present invention more clearly, the following briefly describes the accompanying drawings required for the embodiments. Is should be understood that the following accompanying drawings merely show some embodiments of the present invention, and should not be considered to limit the scope of the present invention.

FIGS. 1A-1C are identification of G4s in the PBX1 locus and the PBX1 transcript. (FIG. 1A) is the schematic flowchart of the bioinformatics workflow of the identification of PQSs in the PBX1 locus and the PBX1 transcript. (FIG. 1B) is G4H, G4NN and cGcC scores of potential G4s in the PBX1 locus and the PBX1 transcript. The G4H, G4NN and cGcC scores of G4s are calculated by the G4RNA screener: G4H, G4hunter; G4NN, G4 neural network; and cccc, cccc, consecutive G vs. consecutive c ratio. (FIG. 1C) is conservation analysis of dG1 candidate species in 100 species. An average PhastCons (100 Vert.) score of the dG1 sequence is obtained from the UCSC genome browser.

FIGS. 2A-2G are features formed by dG1 and rG1 in vitro and in cells. In (FIG. 2A), Left: CD spectra of WT and Mut dG1, and Right: CD spectra of WT and Mut rG1. CD is circular dichroism. In (FIG. 2B), Left: gel mobility assay of WT and Mut dG1, and Right: gel mobility assay of WT and Mut rG1. In (FIG. 2C), under specified conditions, fluorescence-on assay of NMM in absence of presence of dG1 or rG1, and their G/A mutant. In (FIG. 2D), the occupancy of BG4 in the PBX1 promoter region is measured in A375 cells by BG4 chromatin immunoprecipitation (ChIP), followed by qRT-PCR. In (FIG. 2E), RNA immunoprecipitation (RIP) detection is performed to show the association between BG4 and the PBX1 transcript in A375 cells, followed by qRT-PCR. In (FIG. 2F), the occupancy of BG4 in the PBX1 promoter region is measured, by the BG4 chip, in A375 cells treated with or without PDS and TMPyP4, followed by qRT-PCR. In (FIG. 2G), RNA immunoprecipitation (RIP) detection shows that BG4 and the PBX1 transcript are associated in A375 cells treated with or without PDS and TMPyP4, followed by qRT-PCR. Data shows the mean value ±SEM of the three independent experiments, and two-tailed Student's t test. SEM represents the standard error of mean. *P&lt; 0.05, **P&lt; 0.01***P&lt; 0.001.

FIGS. 3A-3J illustrates that PDS and TMPyP4 induce the formation of PBX1 G4s to inhibit the transcription and translation of PBX1. In (FIG. 3A), the activity of g-tetraploid in HEK293T, A375 and B16-F10 cells is measured by luciferase reporter experiments. In (FIG. 3B), the HEK293T, A375 and B16-F10 cells are respectively treated with or without 2 μM PDS or 5 μM TMPyP4. Left panel: a representative confocal image. The scale bar is 100 m. Right: Determination of relative fluorescent values. In (FIGS. 3C-3J), mRNA and protein levels of PBX1 in A375 and B16-F10 cells are detected by qRT-PCR and western blot. PDS or TMPyP4 treatment can inhibit mRNA (FIGS. 3C, 3E, 3G, 3I) and protein levels (FIGS. 3D, 3F, 3H, 3j) of PBX1. From left to right is the protein level of PBX1, and the western blot results are used for statistical analysis. The protein levels of PBX1 are normalized to protein levels of GAPDH.) Data shows the mean value ±SEM of the three independent experiments, and two-tailed Student's t test. *P<0.01, **P<0.01, ***P<0.001.

FIGS. 4A-4P show that PDS and TMPyP4 inhibit growth and lung metastasis of a primary tumor both in vitro and in vivo. In (FIGS. 4A-4B), PDS and TMPyP4 reduce the viability of A375 and B16-F10 cells measured by CCK8. (FIGS. 4C-4H) show the effect of PDS and TMPyP4 on colony formation on cell plates (FIGS. 4C-4D). From left to right: colony formation analysis, and statistical analysis of colony number. From left to right: wound scratch analysis, statistical analysis of wound healing area, and invasion (FIGS. 4G-4H). From left to right: Transwell assay and statistical analysis of invasive cells. Data shows the mean value ±SEM of three independent experiments, and two-tailed Student's t test. (FIGS. 4I-4M) illustrate that PDS inhibits tumor growth. (FIG. 4I) is a representative bioluminescence imaging (BLI) image (five mice per group). (FIG. 4J) illustrates the measurement of the tumor volume on days 0, 7, 14 and 21. (FIG. 4K) illustrates the tumor weight after mouse is sacrificed. In (FIGS. 4L-4M), mRNA and protein levels of PBX1 are measured by qRT-PCR (FIG. 4L) and immunohistochemical (IHC) staining (FIG. 4M). (FIG. 4N) illustrates representative BLI images of the lung tissue of each group. (FIG. 4O) illustrates Violin plot showing BLI quantification of lung metastasis load. In (FIG. 4P), Mice were censored at moribundity, followed by Kaplan-Meier survival curve analysis. n=5 per group, Cox proportional-hazards model, two-sided. n is not important. *P<0.05, **<0.01, ***P<0.001.

FIGS. 5A-5I show that PBX1 rG1 specific ASO inhibits melanoma progression. In (FIG. 5A), in A375 cells, BG4 chip was used to detect the occupancy of BG4 in the PBX1 promoter region, and followed by qRT-PCR. (FIG. 5B) illustrates mRNA levels of PBX1 in A375 and B16-F10 cells treated with or without ASO. (FIG. 5C) illustrates protein levels of PBX1 in A375 and B16-F10 cells treated with or without ASO. From left to right: mRNA and PBX1 protein levels. The protein levels of PBX1 are normalized to protein levels of GAPDH. (FIG. 5D) illustrates PDX clinical sample (left) and the schematic diagram of PDC model (right). (FIG. 5E) illustrates colony formation in A375 and PDC cells. From left to right: colony formation analysis and statistical analysis of colony number. Data shows the mean value ±SEM of the three independent experiments, and two-tailed Student's t test. (FIGS. 5F-5I) illustrate that PDS inhibits tumor growth. (FIG. 5F) illustrates BLI representative image (n=5 per group). (FIG. 5J) illustrates the measurement of the tumor volume on days 0, 7, 14 and 21. (FIG. 5K) illustrates the tumor weight after mouse is sacrificed. In (FIGS. 5H-5I), mRNA and protein levels of PBX1 are measured by qRT-PCR (FIG. 5H) and IHC staining (FIG. 5I). Scale bar: 50 m. **P<0.01, ***P<0.001.

### DETAILED DESCRIPTION

As used herein,
the term “prepared by” is synonymous with the term “comprising”. The terms “comprise”, “comprising”, “include”, and “including” or any other variation thereof, are intended to cover the non-exclusive inclusion. For example, the composition, step, method, product or apparatus that includes a list of features is not necessarily limited only to those features but may include other features not expressly listed or inherent to such composition, step, method, product or apparatus.

The conjunction “consisting of” excludes any element, step or component not identified. If used in a claim, this phrase will make the claim closed such that it excludes materials other than those described, except for usual impurities associated therewith. When the phrase “consisting of” appears in a clause of a claim body rather than immediately after the subject matter, it defines only elements described in the clause; and other elements are not excluded from the claim as a whole.

When amounts, concentrations or other values or parameters are indicated by ranges, preferred ranges or ranges defined by a series of preferred values of upper and lower limits, it should be understood to specifically disclose all ranges formed by any pair of any upper range limit or preferred value and any lower range limit or preferred value, regardless of whether the range is disclosed separately or not. For example, when a range of “1 to 5” is disclosed, the range described should be construed to include ranges of “1 to 4”, “1 to 3”, “1 to 2”, “1 to 2 and 4 to 5”, “1 to 3 and 5” and so on. When a range of numerical values is described herein, unless otherwise specified, the range is intended to include both end values and all integers and fractions within that range.

In embodiments, unless otherwise specified, stated parts and percentages are both measured by mass.

The term “parts by mass” refers to the basic unit of measurement that represents the mass ratio relationship of multiple components. 1 part can represent any unit mass, for example, it can represent 1 g, or 2.689 g and the like. If the parts by mass of component A is a parts and the parts by mass of component B is b parts, then it shows that the mass ratio of component A to component B is a:b. Or, it shows that the mass of the component A is aK, and the mass of the component B is bK (K is a random number, representing the multiple factor). What should not be misunderstood is that the sum of the parts by mass of all the components is not limited to 100 parts, which is different from the mass fraction.

The term of “and/or” is used to refer that one or both of the described conditions may occur, for example, A and/or B includes (A and B) and (A or B).

It should be noted that the type of the reagent is not specifically limited, as long as the specific detection of PBX1 protein expression can be achieved. A person skilled in the art could understand that the term “expression quantity” can represent both an absolute expression quantity and a relative expression quantity.

The technical solutions of the present invention are as follows: In a first aspect of the present invention, provided is an application of an antisense oligonucleotide targeting a PBX1 promoter region G-quadruplex in preparation of medicine for treating melanoma.

Specifically, the medicine for treating melanoma has one or more of the following uses:
inhibiting the proliferation of melanoma cell;
inhibiting the migration of melanoma cell;
inhibiting the invasion of melanoma cell;
inhibiting the growth of melanoma; and
inhibiting the lung metastasis of melanoma.

Optionally, the antisense oligonucleotide targeting a PBX1 promoter region G-quadruplex comprises at least one of the following:
a compound specifically inhibiting PBX1-G4;
an interfering molecule specifically interfering with the expression of PBX1-G4; and
a gene editing reagent specifically knocking out PBX1.

Optionally, the compound specifically inhibiting PBX1-G4 is an antisense oligonucleotide.

Preferably, a content of the antisense oligonucleotide in the medicine is 15 mg/ml to 35 mg/ml.

Optionally, the interfering molecule specifically interfering with the expression of PBX1-G4 is an antisense oligonucleotide.

Preferably, the antisense oligonucleotide has a nucleotide sequence as shown in SEQ ID Nos: 1-2.

In a second aspect of the present invention, provided is a method of screening the medicine, where the medicine is used for preventing or treating melanoma, and the method comprises:
applying candidate medicine to a melanoma model;
quantitatively detecting PBX1 protein of the melanoma model before and after administration; and
indicating the candidate medicine is the target medicine, if an expression level of PBX1 protein in the melanoma model is reduced after the administration, compared with before the administration.

In a third aspect of the present invention, provided is an application of a reagent in preparation of a kit, where the reagent is used for quantitatively detecting an expression level of a PBX1 protein, and the kit is used for determining the effectiveness of medicine in preventing or treating melanoma.

The implementations of the present invention will be described in detail below in conjunction with specific embodiments, but a person skilled in the art will understand that the following embodiments are only used to illustrate the present invention and should not be regarded as limiting the scope of the present invention. Embodiments in which specific conditions are not specified are carried out according to the conventional conditions or conditions suggested by manufacturers. The reagents or instruments employed are all conventional products commercially available if the manufacturer is not indicated.

Animals and reagents used in the embodiments of the present invention are as follows:

Laboratory mice: specific pathogen free (SPF) C57BL/6 mice and BALB/C male nude mice of 4-6 weeks of age used in this study are both purchased from Beijing HFK Bioscience CO., LTD. (Beijing, China). NCG (Cat. #T001475) mice are from Jianyao Pharmaceutical Industry (Jiangsu, China). The mice are raised under a specific aseptic condition for two weeks before the experiment. All in vivo experiments are approved and supervised by the Institutional Animal Care and Use Committee, School of Public Health, Jilin University. All efforts are intended to minimize animal suffering.

Reagents: TMPyP4 (USA Nike; Cat. #P1202) and Pyridostatin (PDS; USA Nike; Cat. #S7444) are dissolved and stored at −80° C. Before the experiment, TMPyP4 and PDS are diluted in culture media to the desired working concentration. All chemicals are used without further purification. pLVX-IRES-mcherry (Article Number: #VT1461), PBX1 promoter pGL3-Basic (Article Number: #VT15541) and pFLAG-CMV 2 (Article Number: #VT1068) are from YouBio (China). Anti-GAPDH (Article Number: #ab9485) and anti-ZIC2 (Cat. #ab150404) are from Abcam in UK. Anti-PBX1 (Cat. #432s) and GAPDH (Article Number: #14793S) are from CST (USA). Anti-PBX1 (Article Number: #PA5-82118) are from Invitrogen (USA). Anti-G4 (Article Number: Ab00174-30.126).

Some experimental methods in the embodiments of the present invention are as follows:

Immunohistochemistry (including IHC) assays: melanoma, liver, spleen, lung, and kidney fixed by Formalin and embedded by paraffin are removed, and then processed into paraffin-embedded tissue blocks. Tissue slices cut from the immunohistochemical blocks are installed on slides, dewaxed in xylene and then subjected to hydration in ethanol. The tissue slices are preprocessed by trypsin (0.1%), and then endogenous peroxidase activity is blocked by using peroxide 1. After 5 min, the tissue slices are stained by a background punisher. Blocked slides are subjected to incubation with antibodies. Then, the slides are subjected to incubation with the rabbit anti-dog horseradish peroxidase secondary antibody. Then, images are captured by using NanoZoomer 2.0-RS digital pathology scanner (Hamamatsu, Japan). Six micrographs are taken in each region.

RNA extraction and quantitative RT-PCR detection: total RNA is extracted from cultured cells by using TRIzol (Invitrogen, USA), then is converted into cDNA by SuperScript III First-Strand Synthesis SuperMix (Invitrogen, USA). qRT-PCR detection is performed by using SYBR Green®Mix (Invitrogen, USA) in ABI 7500 (Invitrogen, USA). A relative expression quantity of RNA is calculated by using a comparative CT method.

Immunoblotting: for protein analysis of a whole-cell lysate, cells are lysed with RIPA (CWBIO, Beijing, China) buffer solution. For tumors, liquid nitrogen is first used to pulverize tissues. Then, the lysate is generated on ice with the RIPA (CWBIO, Beijing, China) buffer solution for 20 minutes, and then centrifuged at 4° C., 13000 rpm for 15 minutes. Protein-containing supernatant is transferred to the fresh microcentrifuge tube, and stored at −20° C. (short-term storage) for subsequent use. The protein is quantified by using the BIO-RAD DC protein assay. The total proteins are electrophoresed on SDS-polyacrylamide gels. Then, the proteins are transferred to polyvinylidene difluoride (PVDF) membrane. After incubation with the primary antibody overnight on the shaker at 4° C., the membrane is washed 6 times (5 minutes each time) with TBS-T (Invitrogen, USA). Then, blots are incubated with horseradish peroxidase (IRP)-conjugated secondary antibodies for 60 minutes at RT on the plate shaker. The membrane is cleaned by TBS-T (6×5 minutes). Detection on immunoreactive bands is performed by using SuperSignalmWest Pico chemiluminescence substrate kit (Thermo Fisher Scientific, USA) and Western blotting detection system (BioRad, USA). GAPDH is used as the loading control for western blotting. The intensity levels of the bands are normalized to GAPDH.

Colony forming assay: for the colony forming assay, A375 and B16-F10 cells are seeded on 6-well plates with the density of 500-1000 cells. After two weeks, staining is performed by using 1% crystal violet at room temperature, rinsing is performed with PBS and tap water, and the number of colonies is counted.

In vitro cell invasion experiment: in the Transwell invasion experiment, Matrigel is used as an invasive supraluminal membrane barrier (Corning, USA). The lower transwell chamber is filled with 2.5% serum containing DMEM medium. The cells are suspended at an upper chamber. After incubation for 24 hours, the filter is removed, and cells on the membrane are fixed by using methanol. Cells stained with 0.5% crystal violet infiltrate the surrounding matrix. The dye is washed with water and the cells are examined under the microscope (Leica, Germany). Six micrographs are taken for each person.

In vivo tumor growth experiment: to evaluate the effect of PBX1 on growth of in vivo tumors, before inoculation, male nude mice of 6 weeks (BALB/C background) are randomly divided into designated groups (5 per group). 1×10⁶ A375 melanoma cells are injected subcutaneously into the upper flank of the back of each mouse. After injection, the mice are administered with PDS drug for treatment for 21 days (once every two days), and after the mice are sacrificed, the tumor weight is measured.

Dual luciferase reporter assay: for the PBX1 luciferase reporter gene assay, PBX1 luciferase reporter gene plasmid and pRL-TK plasmid are co-transfected into HEK293T, A375 and B16-F10 cells in the 96-well plate by Lipofectamine 2000 (Invitrogen, USA). 48 hours after transfection, cells are collected and analyzed using the Dual-Luciferase® Reporter Assay System (Promega, USA). The firefly activity is normalized to the activity of Renilla luciferase.

Construction of an EGFP reporter vector: the reporter vector (Inovogen Tech. Co., Beijing, China) with pLV-EGFP-N as the backbone gene encodes the rG1 site of the PBX1 5′UTR sequence. By using the Strata gene rapid change site-directed mutagenesis kit, point mutation is performed on the rG1 site.

Cell proliferation assay: cell viability is determined by the cell counting kit-8 (CCK-8) according to the manufacturer's instructions (Abcam, UK). Cells (2000 cells per well) are incubated in the 96-well plate in triplicate and then added to each well at various time points at the final concentration of 10%, and incubation is continued at 37° C. After 60 minutes, the absorbance of the sample is measured at the wavelength of 450 nm. Data is analyzed using GraphPad Prism software.

Cell scratch assay: the cells are seeded in 12-well plate and grown for 24 hours to 80% confluency. The bare region is created by the tip across the diameter of the dish. After being treated with PDS (2 μM) and TMPyP4 (5 μM) for 24 h, the cells are washed with PBS and photographed under the microscope (Leica, Germany) to evaluate cell migration. Six micrographs are taken for each person.

In vivo tumor metastasis assay: 2×10⁵ B16-F10 cells are injected into the tail vein of tail vein tumor. According to solutions formulated by the NCI Animal Care and Use Committee, metastatic endpoints are determined for each experiment by maximal primary tumor burden and/or appearance of moribund mice in all treatment groups. PDS (5 mg/kg) is administered twice a day, for 16 consecutive days.

Bioluminescent tumor cell tracking: in vivo whole-animal imaging, anesthetized mice are injected intraperitoneally with 3 mg of d-luciferin (MCE, USA). Luminescence data is collected on an IVIS system (Perkin Elmer, USA). For live tissue imaging, tissues are perfused with PBS, harvested, and incubated in PBS with 1 μg/ml-1d-luciferin. Data analysis is performed using an IVIS Image software package.

PDX model of melanoma: fresh tumor tissues are collected immediately after surgery, placed in RPMI 1640 medium containing penicillin/streptomycin (100 U/ml; 100 μg/ml), fungal zone (1 μg/ml) and Genta Mycin (50 μg/ml) (all from Gibco, USA) (4,5). According to the institutional review and the ethical guidelines for this study approved by the Ethics Committee of the Second Hospital of Jilin University, fresh tumor samples from patients suffering from melanoma are collected for transplantation with informed consent. The mice were kept and all procedures are performed under the approval and supervision of the Institutional Animal Care and Use Committee (IACUC) of the School of Public Health of Jilin University (Changchun, China). Necrotic tissues are excised before transplantation, and tumor tissues are minced to a size of 2×2×2 mm³, and implanted subcutaneously in the flank of 6-week-old NCG male mice to generate first-generation (F1) PDX mice. Successfully transplanted tumor models are passaged and stored using standard methods. H&E staining is used to evaluate the morphology of patient tumor specimens and xenografts of established PDX models. Treatment with ASO Scr or ASO (intravenous injection, 20 mg/kg) is initiated when the tumor volume of the mice reached 220-280 mm³. Tumor growth and body weight measurements are performed. Tumors are dissected at the end of treatment for further processing for RNA, protein and histological analysis.

Preparation of patient-derived tumor cells: tumor tissues of PDXs are used in basic RPMI1640 medium (Gibco, USA. Free-FBS medium). Patient-derived tumor cells (PDC) are prepared from cryopreserved xenograft fragments using the tumor isolation kit (Miltenyi Biotec, Germany), following the tumor treatment solution. PDCs are cultured as described by Bruna and Shanker et al. (6,7). PDCs are used for colony forming assays.

All statistical analyses in the embodiments of the present invention are performed by t test, and expressed as the mean value ±SEM. No animals or samples are excluded from the analysis. p-values are assigned as: *, p<0.05; **, p<0.01; ***, p<0.005; ****, p<0.001; p<0.05 is considered statistically significant.

### Embodiment 1 Identification of G4s Gene in PBX1 Gene Promoter and Transcript

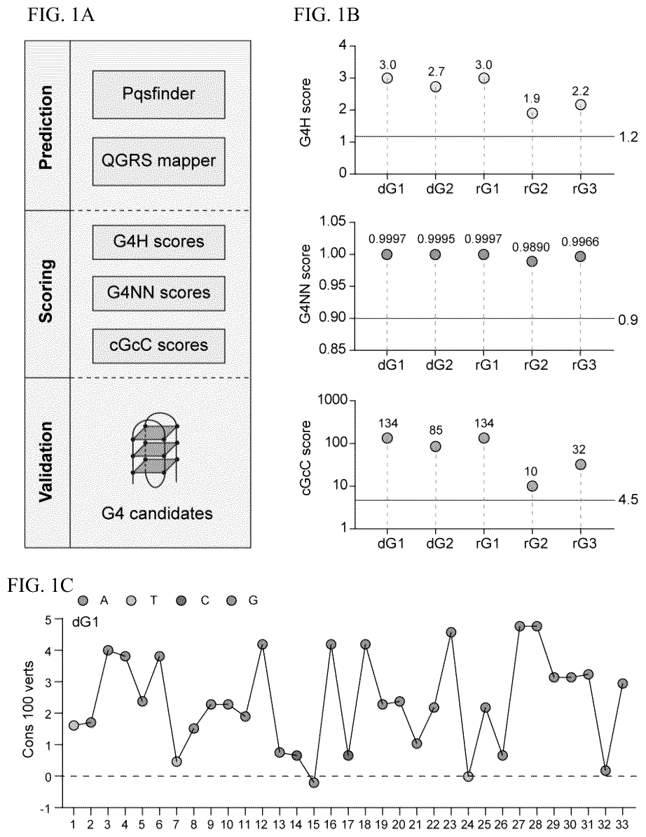

As the PBX promoter and transcript are extremely rich in guanine, the present invention first decides to adopt an integrated strategy to screen for potential G4s in the PBX1 promoter and transcript (FIG. 1A). By combining two independent G4 forecasting softwares, two potential DNA G4s (named dG1 and dG2) are identified from the PBX1 promoter region, and three potential RNA G4s (named rG1, rG2 and rG3) are identified from the PBX1 transcript. In addition, the G4 folding ability of these potential G4s is evaluated by continuous G/C ratio (cGcC), G4Hunter (G4H) and G4 neural network (G4NN) scores. The cGcC, G4H and G4NN scores of dG1 and rG1 are the highest (FIG. 1i), indicating that the G4 forming ability of dG1 and rG1 is the strongest. It is to be noted that in all 100 vertebrate species, the dG1 region is highly conserved, indicating evolutionary functions of dG1 and rG1 in the regulation of PBX1 (FIG. 1C). Therefore, dG1 and rG1 are selected as typical targets for further studies.

### Embodiment 2 Formation of PBX1 G4s In Vitro and in Cells

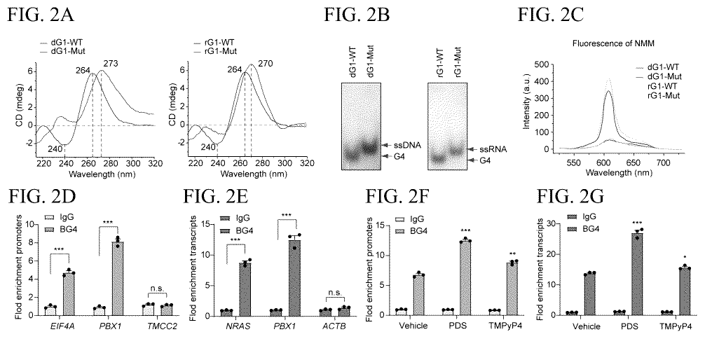

In this embodiment, several methods are further employed to verify the formation of dG1 and rG1. First, circular dichroism (CD) is used to examine the molar ellipticity of dG1 and rG1 sequences (33 nt) and mutants thereof, and the mutants are designed to eliminate G4 formation. In 100 mM K+ buffer solution, the CD spectra of wild-type (WT) dG1 and rG1 sequences show typical 264 nm positive molar ellipsometric peak and 240 nm negative ellipsometric peak, while the mutants do not (FIG. 2A). By gel mobility assay, dG1 and rG1 are found to migrate faster than the mutants thereof, indicating that dG1 and rG1 are folded into the compact secondary structure (FIG. 2B). N-methyl mesoporphyrin IX (NMN) is the well-known fluorescent G4-specific ligand that can be used for studies of G4 formation in vitro. Both dG1 and rG1 are found to enhance the fluorescence of N N (FIG. 2C). In addition, in this embodiment, chromatin immunoprecipitation (ChIP) and RNA immunoprecipitation (RIP) detection are performed in A375 cells using the well-known G4-specific antibody BG4. It is found that the PBX1 promoter and transcript could be pulled down by BG4 (FIG. 2D and FIG. 2E), indicating that PBX1 G4s ARE formed in the cells. In summary, dG1 and rG1 can fold stable G4 structures in vitro and in cells. G4-specific ligands, including PDS and TMPyP4, etc., have been proved to have the ability to bind and stabilize DNA and RNA G4s. Therefore, the ability of these G4-specific ligands to interact with PBX1 G4s is investigated next. It is found that both PDS and TMPyP4 remarkably enhance the thermal stability of dG1 and rG1, indicating that PBX1 G4s has the strong interaction with the two g4-specific ligands. In addition, ChIP and RIP analyses show that PDS and TMPyP4 can enhance pull-down of the PBX1 promoter and BG4 transcript (FIG. 2F and FIG. 2G). In conclusion, PDS and TMPyP4 can bind and stabilize PBX1 G4s.

### Embodiment 3 Formation of PBX1 G4s Inhibits Transcription and Translation of PBX1

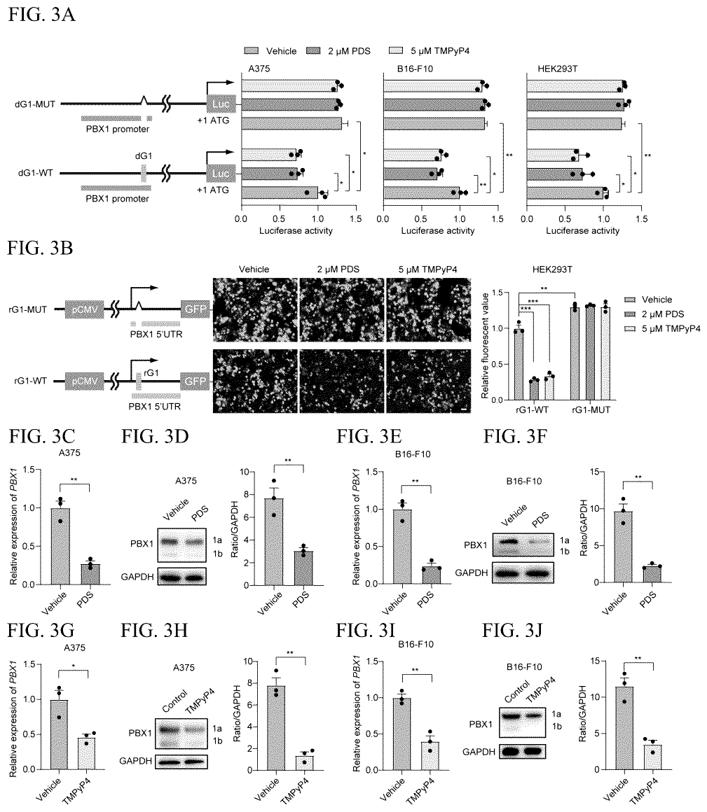

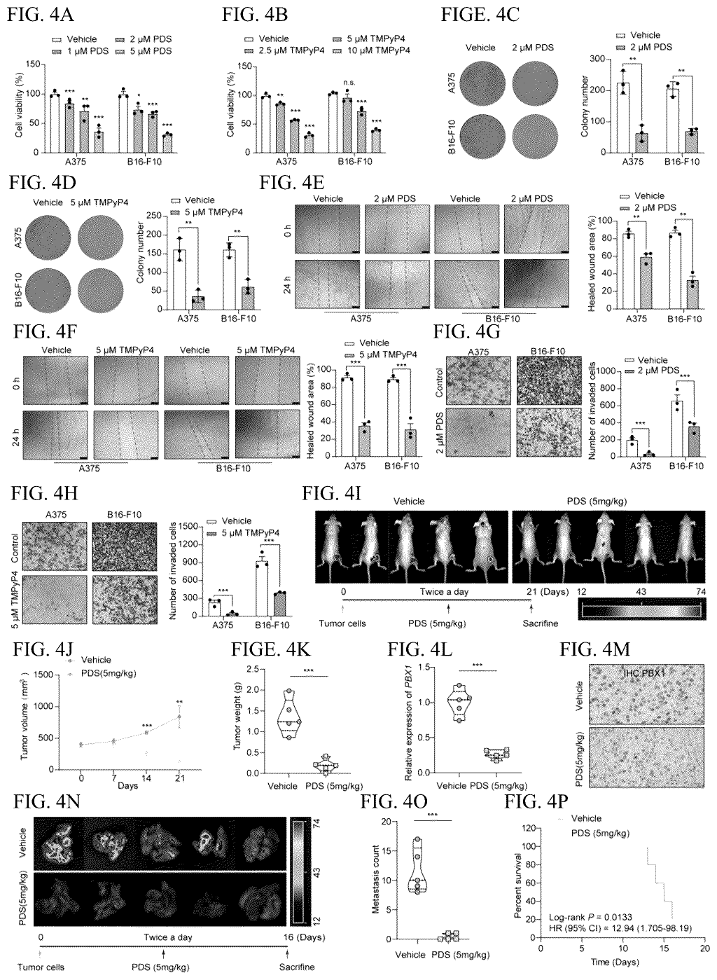

Given that G4s in the promoter and transcript are involved in the regulation of transcription and translation related processes, it is speculated that dG1 in the PBX1 promoter may regulate the transcription of PBX1, and rG1 in 5′UTR of the PBX1 transcript may regulate the transcription of PBX1. In addition, the luciferase reporter assay is first performed. The dG1 promoter sequence (designated as dG1-wt) and mutated dG1 promoter sequence (designated as dG1-mut) of PBX1 are cloned into pGL3 luciferase reporter vector. Compared with dG1-MUT transfected cells, the luciferase activity of dG1-WT transfected HEK293T, A375 and B16-F10 cells is remarkably reduced (FIG. 3A). It is to be noted that PDS and TMPyP4 treatment remarkably inhibits the luciferase activity of dG1-wt transfected cells, but does not affect dG1-mut transfected cells (FIG. 3A), indicating that the G4-specific ligands inhibit the transfection process by targeting PBX1 dG1. In addition, this embodiment further constructs an enhanced green fluorescent protein (EGFP) reporter gene system, to study the effect of rG1 on translation. The rG1 transcript sequence (designated as rG1-wt) and mutant rG1 transcript sequence (designated as rG1-mut) of PBX1 are fused to 5′UTR of EGFP, and the reporter gene vector is transfected into HEK293T cells. By confocal fluorescence experiments, the EGFP fluorescence intensity of rG1-WT is less than that of rG1-MUT, and PDS or TMPyP4 decreases the fluorescence intensity of rG1-WT in HEK293T cells (FIG. 3B). Finally, this embodiment studies the potential effect of PBX1 G4s on PBX1 expression of A375 and B16-F10 cells. It is found that PDS and TMPyP4 treatment remarkably reduces the mRNA and protein levels PBX1 (FIG. 3C to FIG. 4J). In conclusion, PDS and TMPyP4 induce formation of PBX1 G4s, to inhibit expression of PBX1.

### Embodiment 4 Formation of PBX1 G4s Inhibits Melanoma Progression by Downregulation of PBX1

This embodiment studies whether formation of PBX1 G4s inhibits melanoma progression by downregulation of PBX1. First, the effects of PDS and TMPyP4 on melanoma cell lines are examined. It is observed that PDS and TMPyP4 treatment remarkably reduces the activity of A375 and B16-F10 cells in the dose-dependent manner (FIG. 4A and FIG. 4B). Similarly, PDS and TMPyP4 treatment inhibits the colony formation of A375 and B16-F10 cells (FIG. 4C and FIG. 4D). PDS and TMPyP4 also inhibit the migration and invasion of A375 and B16-F10 cells (FIG. 4E to FIG. 4H). The effect of PDS on tumorigenicity is further tested in mice. Consistent with the in vitro results, PDS treatment remarkably inhibits growth and metastasis of xenograft tumors by downregulating expression of PBX1 (FIG. 4I to FIG. 4P). In conclusion, these results indicate that formation of PBX1 G4s induced by PDS and TMPyP4 inhibits melanoma progression by downregulation of PBX1.

### Embodiment 5 PBX1 ASO Inhibits Melanoma Progression by Specifically Targeting rG1

Although PDS and TMPyP4 show the strong anti-tumor effect on melanoma, the anti-tumor effects thereof may be partly due to binding to other cellular G4s, and an off-target effect may also lead to side effects, limiting potential for clinical application in melanoma therapy. Antisense oligonucleotides (ASOs) have good potential for clinical application, and are used for inducing formation of RNA G4s. The RNA G4 structure is the motif that is part of the larger RNA context, and folding thereof is affected by the neighbor sequence, because G4s and the neighbor sequence can participate in the Watson-Crick-based stable structure. Therefore, an ASO specific for the complementary sequence of rG1 in the 5′ UTR of PBX1 is designed, inducing formation of PBX1 rG1 (ASO PBX1-G4:

mG*mG*mG*G*T*mT*mG*mC*mG*mG*mG*mG*mT*mG*mA*G*G*G*
T*mG*; ASO

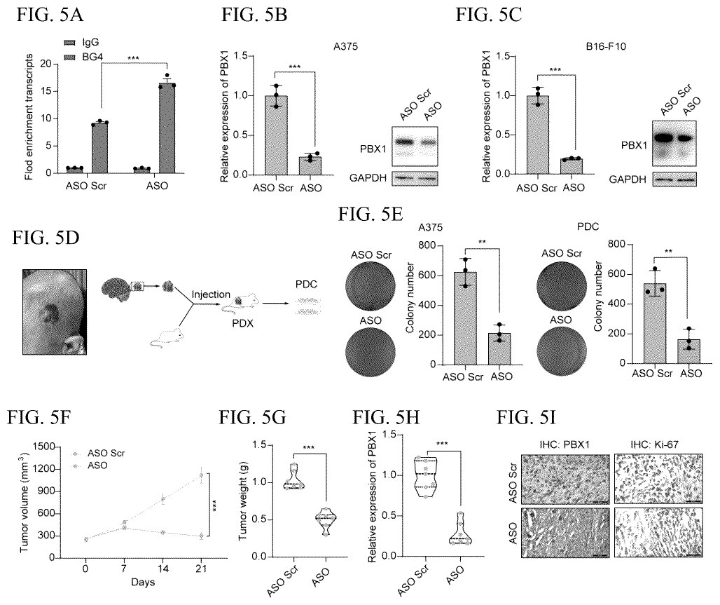

Control:mG*mT*mC*mC*mA*mC*mA*mA*mA*mC*mA*mC*mA*mA*mC*mT*mC* mC*mT*mG*). In reality, RIP detection shows that ASO treatment increases the PBX1 transcript pulled down by BG4 (FIG. 5A), indicating that ASO induces formation of rG1. In addition, ASO treatment of A375 and B16-F10 melanoma cell lines can effectively reduce PBX1 protein and mRNA abundance (FIG. 5B and FIG. 5C). This example also establishes short-term cultures of patient-derived melanoma xenograft (PDX)-derived tumor cells (PDCs) (FIG. 5D). Therefore, the colony formation experiments of A375 cells and PDCs show that the colony formation ability decreases after ASO treatment (FIG. 5E). To further expand the analysis, this embodiment establishes the PDX model of melanoma, and PBX1 is administered intravenously (intravenous injection) every two days for 21 days. Compared with tumors treated by PBX1 ASO scramble, significant reduction in ASO-treated tumor growth and weight is observed (FIG. 5F and FIG. 5G). Moreover, ASO treatment results in significant downregulation of expression of PBX1 and proliferation marker Ki67 (FIG. 5H and FIG. 5I). In conclusion, these results show that ASO-induced PBX1 rG1 formation is the promising therapeutic strategy against melanoma.

Finally, it should be noted that the foregoing embodiments are merely used for describing the technical solutions of the present invention, rather than limiting the present invention. Although this application is described in detail with reference to the foregoing embodiments, persons of ordinary skill in the art should understand that they can still make modifications to the technical solutions described in the foregoing embodiments or make equivalent replacements on some or all of technical features therein. These modifications or replacements do not cause the essence of the corresponding technical solutions to depart from the scope of the technical solutions of the embodiments of the present invention.

In addition, the person skilled in the art would understand that although some embodiments herein include certain features included in other embodiments but not other features, combinations of features from different embodiments are meant to fall within the scope of the present invention and form different embodiments. For example, in the description above, any one of the claimed embodiments may be used in any combination. The information disclosed in this Background section is only for enhancement of understanding of the general background of the present invention and should not be taken as an acknowledgment or any form of suggestion that this information forms the prior art already known to the person skilled in the art.

1. An application of an antisense oligonucleotide targeting a PBX1 promoter region G-quadruplex in a preparation of a medicine for treating melanoma.

2. The application of the antisense oligonucleotide targeting the PBX1 promoter region G-quadruplex in the preparation of the medicine for treating melanoma according to claim 1, wherein the medicine for treating melanoma has one or more of following uses:
inhibiting a proliferation of melanoma cell;
inhibiting a migration of melanoma cell;
inhibiting an invasion of melanoma cell;
inhibiting a growth of melanoma; and
inhibiting a lung metastasis of melanoma.

3. The application of the antisense oligonucleotide targeting the PBX1 promoter region G-quadruplex in the preparation of the medicine for treating melanoma according to claim 2, wherein the antisense oligonucleotide targeting the PBX1 promoter region G-quadruplex comprises at least one of followings:
a compound specifically inhibiting PBX1-G4;
an interfering molecule specifically interfering with an expression of PBX1-G4; and
a gene editing reagent specifically knocking out PBX1.

4. The application of the antisense oligonucleotide targeting the PBX1 promoter region G-quadruplex in the preparation of the medicine for treating melanoma according to claim 3, wherein the compound specifically inhibiting PBX1-G4 is an antisense oligonucleotide.

5. The application of the antisense oligonucleotide targeting the PBX1 promoter region G-quadruplex in the preparation of the medicine for treating melanoma according to claim 4, wherein a content of the antisense oligonucleotide in the medicine is 15 mg/ml to 35 mg/ml.

6. The application of the antisense oligonucleotide targeting the PBX1 promoter region G-quadruplex in the preparation of the medicine for treating melanoma according to claim 3, wherein the interfering molecule specifically interfering with the expression of PBX1-G4 is an antisense oligonucleotide.

7. The application of the antisense oligonucleotide targeting the PBX1 promoter region G-quadruplex in the preparation of the medicine for treating melanoma according to claim 6, wherein the antisense oligonucleotide has a nucleotide sequence as shown in SEQ ID Nos: 1-2.

8. A method of screening a medicine, wherein the medicine is used for preventing or treating melanoma, and the method comprises:
applying a candidate medicine to a melanoma model;
quantitatively detecting PBX1 protein in the melanoma model before and after administration; and
indicating the candidate medicine is a target medicine, if an expression level of PBX1 protein in the melanoma model is reduced after the administration, compared with before the administration.

9. An application of a reagent in a preparation of a kit, wherein the reagent is used for quantitatively detecting an expression level of PBX1 protein, and the kit is used for determining an effectiveness of medicine in preventing or treating melanoma.


SEQUENCE LISTING (2 sequences)

SEQ ID NO: 1 - RNA, 20 nt, synthetic construct
  misc_feature 1..20: note = Synthesized
  source 1..20: mol_type = other RNA
ggggttgcggggtgagggtg

SEQ ID NO: 2 - RNA, 20 nt, synthetic construct
  misc_feature 1..20: note = Synthesized
  source 1..20: mol_type = other RNA
gtccacaaacacaactcctg